(19) United States
(12) Patent Application Publication
(10) Pub. No.: US 2016/0015914 A1
(43) Pub. Date: Jan. 21, 2016

(54) MEDICAMENT DELIVERY DEVICES

(71) Applicant: INNOVATA BIOMED LIMITED, Edinburgh (GB)

(72) Inventor: Philip Carl PARKES, Edinburgh (GB)

(21) Appl. No.: 14/378,600

(22) PCT Filed: Feb. 5, 2013
(86) PCT No.: PCT/GB13/50263

§ 371 Date: Aug. 13, 2014

(30) Foreign Application Priority Data
Feb. 21, 2012 (GB) 1202938.5

(51) Int. Cl. A61M 15/00

(52) U.S. Cl. CPC A61M 15/0003; A61M 15/0065; A61M 15/0006; A61M 2202/064

(57) ABSTRACT

A medicament delivery device (1) comprises a metering member (25) mounted for rotation adjacent a reservoir (22) containing a bulk quantity of medicament. The metering member (25) is adapted when at a filling position to volumetrically meter a desired dose of medicament from the reservoir (22). By rotation of the metering member (25) in a first sense said dose is transferred to a dispensing position at which the dose can be dispensed from the device (1). The metering member (25) and/or a component (90) to which the metering member (25) is operably linked is provided with formations (95) effective to prevent reverse rotation of the metering member (25) from the filling position.

This invention relates to improvements to medicament delivery devices, and in particular to medicament delivery devices in which a unit dose of medicament is transferred from a bulk reservoir by a rotary transfer mechanism. The invention is of particular utility in relation to devices for the administration of medicaments by inhalation, especially dry powder inhalers (DPIs).

The administration of medicaments by inhalation is well-known. A wide variety of medicaments are now administered by that route, for the treatment of a range of respiratory disorders.

The most common form in which such medicaments are formulated for administration by inhalation is as a powder. In the past, many such compositions were formulated as pressurised aerosols, in which the powder medicament was suspended in a liquefied propellant. Due to the adverse environmental effects of the propellants conventionally used, however, there is now increased interest in the use of DPIs. In a DPI, a unit dose of medicament powder, either packaged as such or metered from a bulk reservoir of medicament, is presented to an airway and is then entrained in an airflow passing through the airway. The airflow is most commonly generated by the patient's act of inhalation.

The CLICKHALER® DPI produced by Innovata Biomed Ltd in the UK includes a metering mechanism that is described in EP0539649B. In that device, a metering member is rotatably mounted beneath, and in close abutment with, an opening at the base of a reservoir containing a bulk quantity of powdered medicament. The metering member is formed with a series of depressions that serve as volumetric metering cups. When a cup is in registration with the opening in the reservoir (the filling position), the cup fills with medicament (a unit dose). Rotation of the metering member displaces the cup from that position to a position from where the unit dose can be inhaled (the dispensing position), at the same time bringing the next cup to the filling position. Thus, by indexed rotation of the metering member, the metering cups are successively moved to the filling position, and thereafter to the dispensing position.

WO01/39823 describes another DPI that functions in a similar manner, save that in this device there are two reservoirs containing different medicaments that are to be administered together. Each reservoir is associated with a rotating metering member.

WO2011/007181 describes an actuation mechanism for such a DPI, in which the two metering members are coupled to a common drive wheel that is acted upon by a reciprocating actuator. The actuator is in turn driven by a push-button that is depressed by the user. Each depression of the push-button causes the drive wheel to be rotated, and metering cups on the two metering members to be displaced from their filling positions to their dispensing positions.

DPI devices of the type described above have proven to be very successful. However, it has now surprisingly been found that further improvements in performance, and in particular improvements in the accuracy of the volumetric dosing of the medicament(s) and hence improvements in the uniformity of the dose administered to the user, can be brought about by measures intended to prevent reverse rotation of the metering member(s).

Thus, according to the invention, there is provided a medicament delivery device comprising a metering member mounted for rotation adjacent a reservoir containing a bulk quantity of medicament, the metering member being adapted when at a filling position to volumetrically meter a desired dose of medicament from the reservoir and by rotation of the metering member in a first sense to transfer said dose to a dispensing position at which the dose can be dispensed from the device, the metering member and/or a component to which the metering member is operably linked being provided with formations effective to prevent rotation of the metering member from the filling position in the sense opposite to the first sense, the formations effective to prevent rotation of the metering member from the filling position in the sense opposite to the first sense constituting a ratchet-type mechanism, the metering member and/or the component to which the metering member is operably linked on the one hand and an adjacent surface of the device on the other hand being provided with ratchet and pawl formations, wherein the metering member and/or the component to which the metering member is operably linked is provided with one or more pawls that engage, when the metering member is in the filling position, with corresponding ratchet formations to prevent reverse rotation of the metering member.

The medicament delivery device according to the invention is advantageous primarily in that because rotation of the metering member in the reverse sense is prevented, small displacements of the metering member from the filling position, which might otherwise be caused by forces acting on the metering member as a result of operation of the device (e.g. the return of an actuator to its rest position), are eliminated. This may improve the precision of the volumetric dosing of medicament from the reservoir and so improve the uniformity of the dose received by the user of the device.

The medicament delivery device may be an inhaler, and in particular may be a DPI. However, the invention may find utility in other forms of medicament delivery device, e.g. nasal spray devices and the like.

The device may be used to dispense unit doses of a single medicament.

Alternatively, the device may be used to dispense unit doses of a formulation containing two or more medicaments. In a further alternative, the device may be used to dispense unit doses of two or more medicaments from separate reservoirs within the device, those unit doses then being administered to the patient simultaneously.

In many embodiments of the device according to the invention, operation of the device is brought about by means of a push-button or the like, which acts upon an actuator that undergoes a reciprocating motion. By “reciprocating motion” is meant displacement in one direction along a path and then return in the opposite direction along the same path. The path may be linear or have some other suitable form, e.g. the path may be arcuate or radiussed.

The actuator may act directly on the metering member. Alternatively, the actuator may act upon a component that is operably linked to the metering member. The latter arrangement may be particularly convenient where the device contains more than one (most commonly, two) reservoirs of medicament containing different medicaments that are to be administered together. In such a case, each reservoir will generally be associated with a separate metering member, and the metering members may be coupled to a drive component such that rotation of the drive component causes rotation of the metering members.

Where the device contains two reservoirs of medicament, with first and second metering members, the drive component may have the form of a wheel mounted between the first and second metering members for rotation about a common axis. The first and second metering members may be coupled to the wheel in the manner described in WO2011/007181, i.e. by being provided with sockets that receive the respective ends of a compression spring, the sockets having extensions that project into an axial bushing of the wheel, which bushing in turn is provided on its internal surface with formations that engage the extensions to cause the metering members to rotate when the wheel is rotated.

Rotation of the wheel is preferably brought about by engagement of the actuator with suitable formations provided on the wheel. Such formations typically take the form of a plurality of lugs that are equiangularly spaced around the wheel. In such an arrangement, the tip of the actuator may lie, in its rest position, adjacent to one such lug, so that downward movement of the actuator drives the lug downwards, and causes the wheel to rotate. Typically, there will be six or eight such lugs, in which case the length of the stroke of the actuator is chosen such that it causes the wheel to rotate through 60° or 45° respectively. The actuator then returns to its rest position, at which point it bears against the next lug on the wheel. The leading surface of the lug may be ramped in order to facilitate return of the actuator to its rest position, the tip of the actuator riding over the following lug on its return stroke.

In a currently preferred embodiment, a metering member is associated with such a drive wheel. The drive wheel is provided with one or more pawls at its periphery. Most preferably, the number of pawls is greater than one, most commonly two or four, particularly two. Where there is a plurality of pawls, they are preferably equiangularly spaced. The pawls are preferably resilient and extend slightly from the circumference of the wheel. A number of ratchet formations are formed in a surface of the device adjacent to the periphery of the wheel. Usually, the ratchet formations correspond in number to the number of indexed movements of the metering member that are required to cause one full rotation. So, for instance, if the metering member is formed with eight metering cups having an angular separation of 45° and hence eight actuations of the device are required to cause one full rotation of the metering member, there will be eight ratchet formations. The arrangement is such that whenever the metering member is rotated such that a metering cup occupies a filling position, the pawls engage with a corresponding number of ratchet formations to prevent reverse rotation of the metering member.

As the drive wheel is rotated, the resilience of the pawls allows them to deflect inwardly and so permit rotation. When the wheel has been rotated sufficiently that the next metering cup comes into registration with the opening at the base of its associated reservoir (the filling position), the pawls engage with ratchet formations.

In a currently preferred embodiment, the drive wheel rotates within a generally circular recess formed in a support component that holds the metering member. The ratchet formations are formed in the wall of the circular recess, which is spaced from the edge of the wheel sufficiently to permit rotation in the first sense but is sufficiently close to the wheel to permit engagement of the pawl(s) with the ratchet formations whenever a metering cup is moved into the filling position.

Where it is the metering member itself that is provided with formations effect to prevent reverse rotation, rather than a component to which the metering member is operably linked, the arrangement may be analogous to that described above, i.e. the metering member itself may carry one or more pawls at its periphery that engage with ratchet formations in a surrounding surface.

The medicament delivery device is preferably an inhaler, and more preferably a DPI, such as the CLICKHALER® inhaler produced by Innovata Biomed Ltd in the UK and described in EP0539469B or the combination therapy DPI described in WO01/39823.

A variety of medicaments may be administered using the inhaler of the invention.

Such medicaments are generally suitable for the treatment of asthma, COPD and respiratory infections. Such medicaments include, but are not limited to β2-agonists, eg fenoterol, formoterol, pirbuterol, reproterol, rimiterol, salbutamol, salmeterol and terbutaline; non-selective beta-stimulants such as isoprenaline; xanthine bronchodilators, eg theophylline, aminophylline and choline theophyllinate; anticholinergics, eg ipratropium bromide, oxitropium and tiotropium; mast cell stabilisers, eg sodium cromoglycate and ketotifen; bronchial anti-inflammatory agents, eg nedocromil sodium; and steroids, eg beclomethasone, fluticasone, budesonide, flunisolide, triamcinolone, mometasone and ciclesonide; and/or salts or derivatives thereof.

Specific combinations of medicaments which may be mentioned include combinations of steroids and β2-agonists. Examples of such combinations are beclomethasone dipropionate and formoterol; beclomethasone dipropionate and salmeterol; fluticasone and formoterol; fluticasone and salmeterol; budesonide and formoterol; budesonide and salmeterol; flunisolide and formoterol; flunisolide and salmeterol; ciclesonide and salmeterol; ciclesonide and formoterol; mometasone and salmeterol; and mometasone and formoterol.

Further medicaments which may be mentioned include systemically active materials, such as proteinaceous compounds and/or macromolecules, for example hormones and mediators, such as insulin, human growth hormone, leuprolide and alpha interferon, growth factors, anticoagulants, immunomodulators, cytokines and nucleic acids.

One embodiment of the invention will now be described in greater detail, by way of illustration only, with reference to the accompanying drawings, in which:

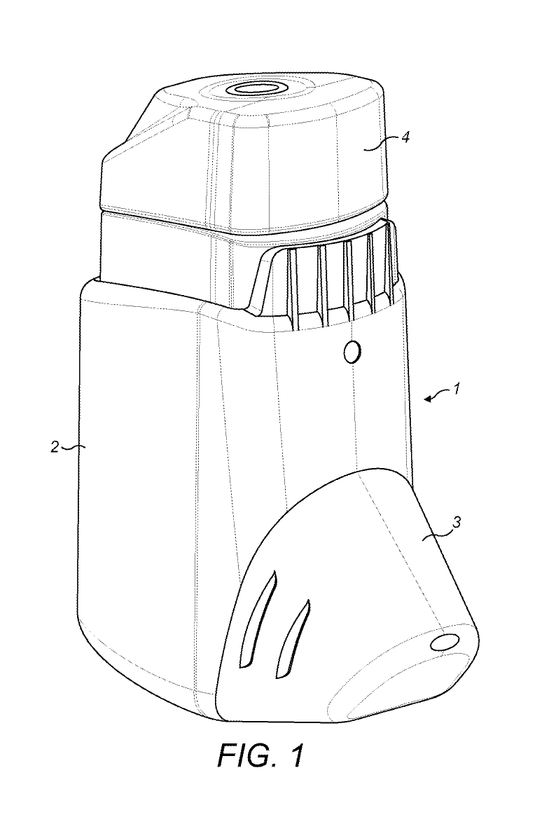

FIG. 1 is a perspective view of a dry powder inhaler device according to the invention;

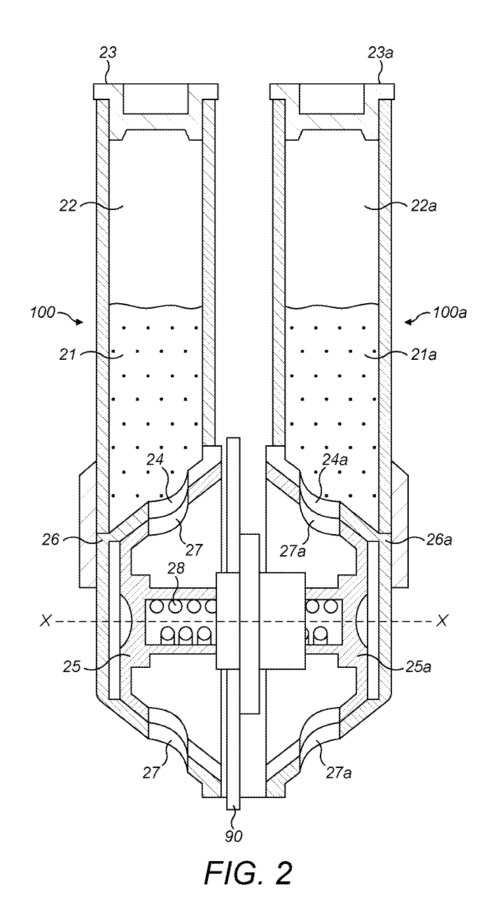

FIG. 2 is a simplified sectional view of the metering mechanism of the dry powder inhaler of the present invention;

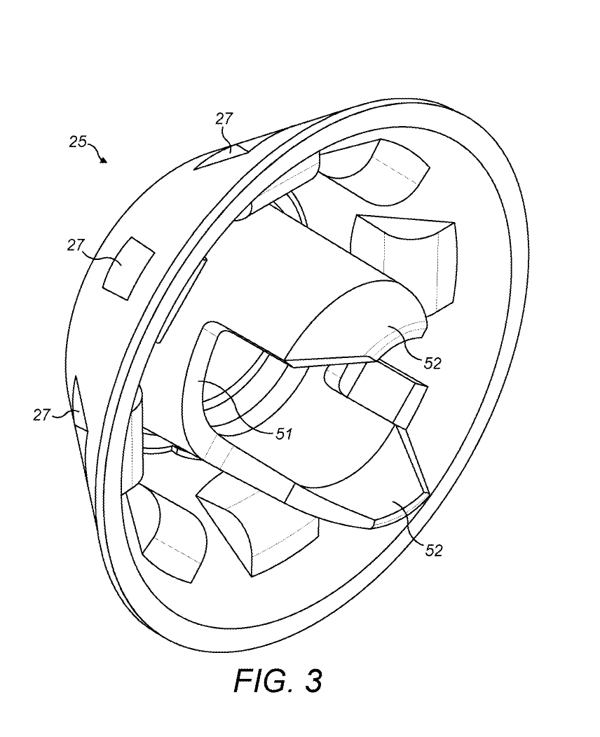

FIG. 3 is a perspective view of a metering member forming part of the dry powder inhaler of the invention;

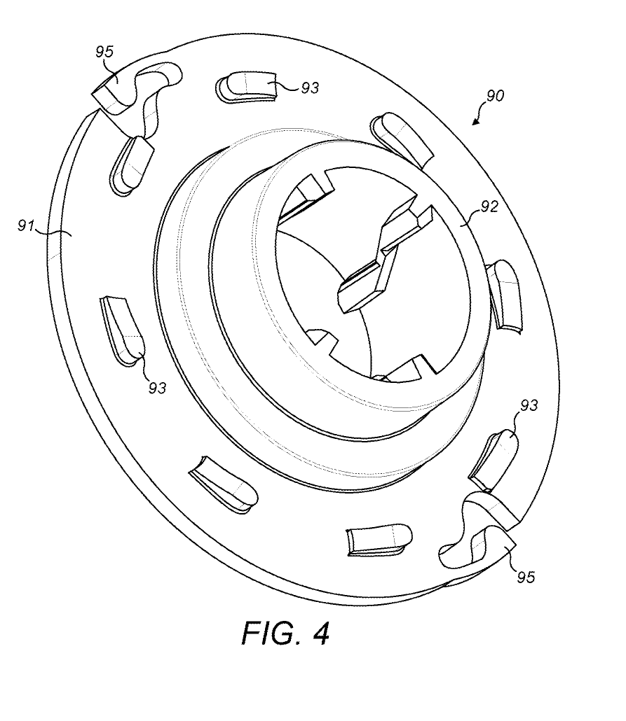

FIG. 4 is a perspective view of a wheel that engages a pair of metering members of the form shown in FIG. 3 in the dry powder inhaler of the invention;

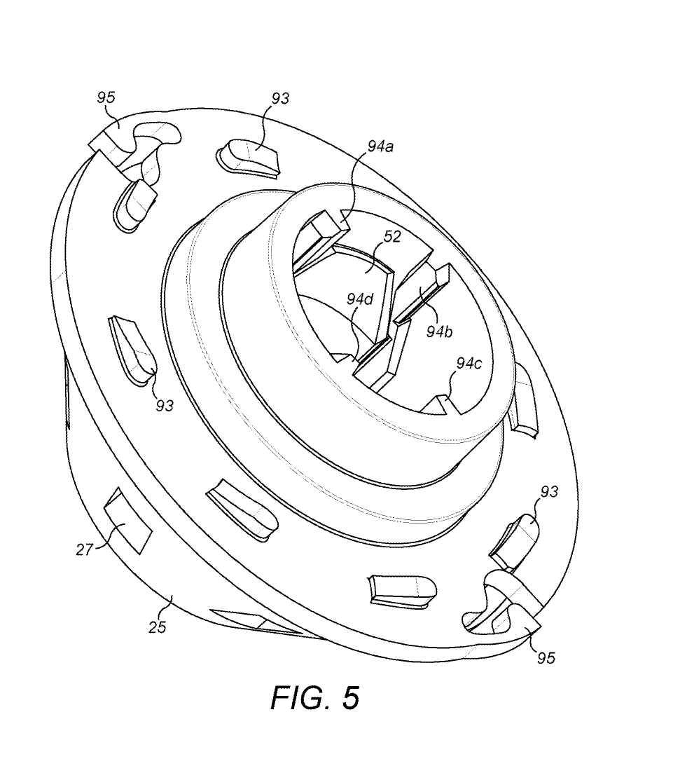

FIG. 5 shows the wheel of FIG. 4 engaged with the metering member of FIG. 3;

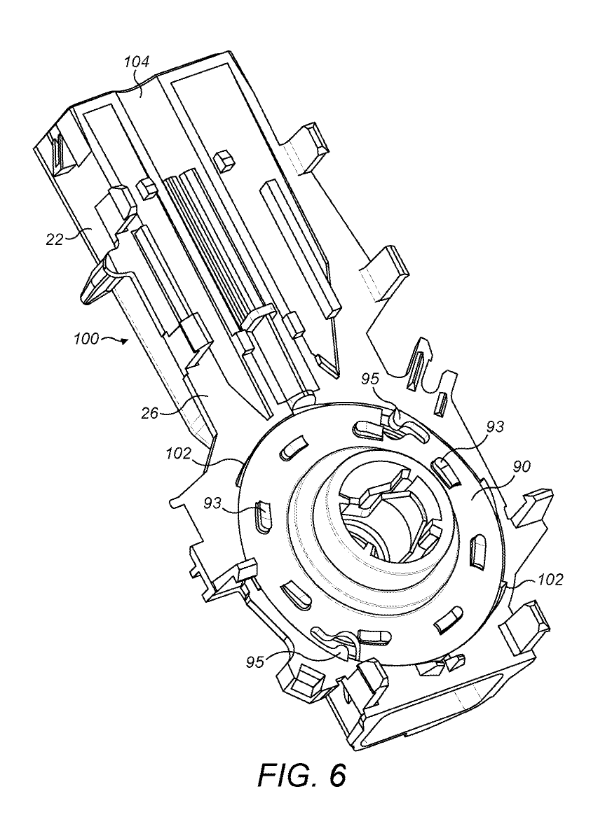

FIG. 6 shows the assembly of wheel and metering member of FIG. 5 located within a support member that incorporates a medicament reservoir;

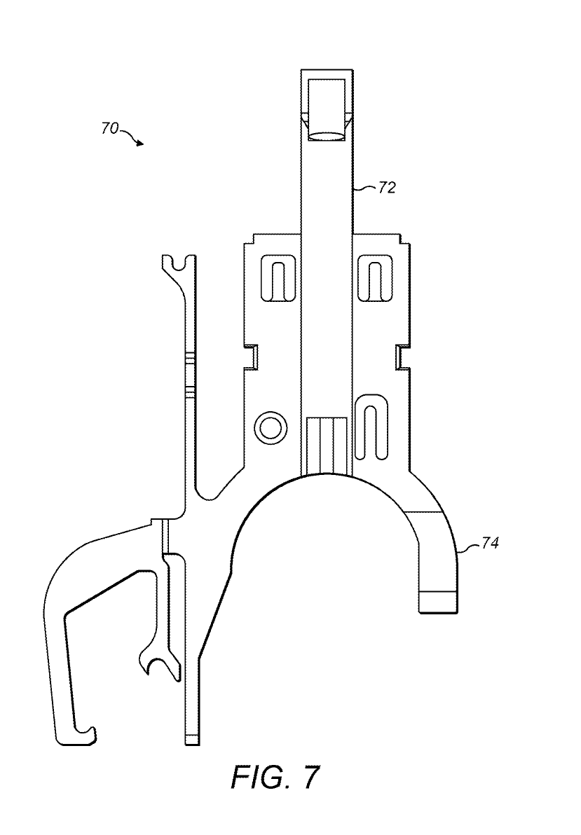

FIG. 7 is a side elevation of an actuator that, in use, acts upon the wheel of FIG. 4; and

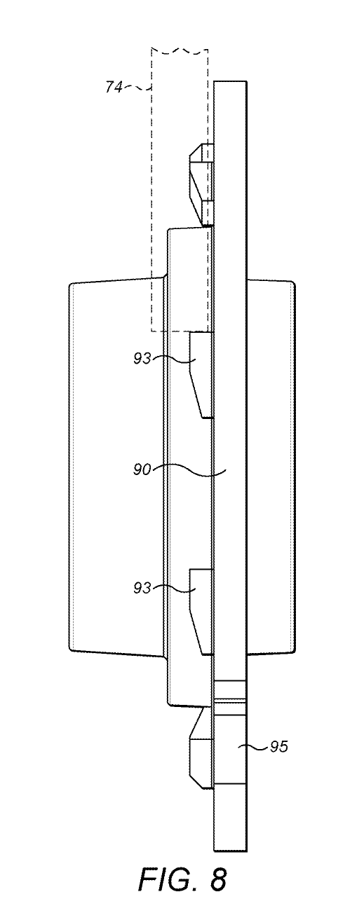

FIG. 8 is a side view of the wheel of FIG. 4, showing the manner in whish the wheel is engaged by the actuator of FIG. 7.

Referring first to FIG. 1, a dry powder inhaler is generally designated 1 and comprises a body 2 with an integral mouthpiece that is covered by a removable cap 3. The upper (as viewed in FIG. 1) part of the inhaler 1 comprises a depressible push-button 4.

The inhaler 1 is for the simultaneous administration of unit doses of two different powdered medicaments, and includes a metering mechanism that is broadly as described in EP1233805B and is shown in somewhat simplified form in FIG. 2.

Referring to FIG. 2, the inhaler 1 includes first and second reservoirs 22, 22a that contain bulk quantities of the two medicaments 21, 21a. The reservoirs 22, 22a are arranged side by side and, in normal use of the inhaler 1, substantially vertically. The upper ends of the reservoirs 22, 22a are closed by caps 23,23a.

The reservoir 22 has an opening 24 in its base, from which powdered medicament 21 is discharged from the reservoir 22 under the influence of gravity. A frustoconical metering member 25 is positioned beneath the reservoir 22, such that the surface of the metering member 25 closely abuts the underside of the reservoir 22 so as to close off the opening 24. The reservoir 22 is formed integrally with a frustoconical recess 26 within which the metering member 25 is received. The metering member 25 has a number of depressions 27 formed in its surface. Two such depressions 27 are evident in FIG. 2. The depressions 27 serve as metering cups by which a dose of medicament 21 is volumetrically dispensed from the reservoir 22. When a depression 27 is in registration with the opening 24, the depression 27 fills with a dose of powdered medicament 21. Rotation of the metering member 25 then transports that dose to a position from which it can be inhaled by the patient, and at the same time brings another depression 27 into registration with the opening 24.

The other reservoir 22a and associated components are identical in all material respects to those described in the preceding paragraph, save that they are disposed in an arrangement that is the mirror image of that depicted for the first reservoir 22. Components associated with the second reservoir 22a that correspond to components 24, 25, 26, 27 associated with the first reservoir 22 are denoted 24a, 25a, 26a, 27a.

The two metering members 25, 25a are each coupled to a wheel 90 (as described below), and are capable of rotating about a common axis, indicated in FIG. 2 by the broken line X-X.

A compression spring 28 is mounted between the metering members 25, 25a, and urges the metering members 25, 25a outwards, into abutment with the respective supports 26, 26a.

The general manner of operation of the inhaler 1 is that depressions 27, 27a that are in registration with the openings 24, 24a are charged with doses of the respective medicaments 21, 21a. When it is desired to administer these doses, the patient causes the metering members 25, 25a to rotate such that the doses are transported to a position from which they can be inhaled. At the same time, empty depressions 27, 27a are brought into registration with the openings 24,24a. Rotation of the metering members 25, 25a is brought about by a ratchet-type mechanism operated by depression of the push button 4, as described below.

The metering member 25 of the inhaler 1 is shown in detail in FIG. 3. As can be seen, the metering member 25 has a generally frustoconical form. The conical part of the outer surface of the metering member 25 is formed with a plurality of depressions (metering cups) 27, that are spaced equiangularly around the metering member 25. In the illustrated embodiment, the metering member 25 has eight such depressions 27.

The underside of the metering member 25 is hollow and is formed with a circular socket 51, from which a pair of diametrically opposed extensions 52 project. The internal diameter of the socket 51 is such that it closely receives the compression spring 28, as described in more detail below. The extensions 52 project beyond the frustoconical part of the metering member 25 and are of arcuate cross-section, being in effect extensions of the circular wall of the socket 51. Each projection 52 has a width that corresponds to a little less than 90° of the circumference of the socket 51. The tips of the projections 52 are tapered.

FIG. 4 shows a wheel 90 that couples the operation of an actuator (shown in FIG. 7 and described more fully below) to the metering members 25, 25a. The wheel 90 comprises a disc 91 with a central cylindrical bushing 92. One face of the disc 91 is formed with a plurality of lugs 93. Each lug 93 has a flat top and a ramped part that extends in the direction of rotation of the wheel 90 (as described below). In the illustrated embodiment, there are eight lugs 93.

As can be seen in FIG. 4, the wheel 90 is formed with a pair of pawls 95 that extend a short distance outwardly of the circumference of the remainder of the wheel 90. The wheel 90 is formed by injection moulding in plastics material, with the result that the pawls 95 are resiliently deformable from their rest position (depicted in FIG. 4) towards the centre of the wheel 90. To permit flexing movement of the pawls 95, the areas of the disc 91 adjacent to the pawls 95 are cut away with shapes broadly corresponding to that of the pawls 95.

FIG. 5 shows the metering member 25 of FIG. 3 engaged with the wheel 90. As can be seen in both FIGS. 4 and 5, the internal surface of the bushing 92 is formed with four splines 94a-d that extend along the full length of the bushing 92, generally parallel to the longitudinal axis of the bushing. The splines 94a-d have a dog-leg or cranked configuration. The effect of this is that, at the end of the bushing 92 that is shown in FIGS. 4 and 5, the ends of the splines 94a, 94b are separated by an angle of less than 90° and the same is true of the ends of the splines 94c, 94d. The angle between the pairs of splines 94b, 94c and between the splines 94d, 94a, on the other hand, is slightly greater than 90°. At the other end of the bushing 92, it is the pairs of splines 94b, 94c and 94a, 94d that are separated by less than 90°.

The extensions 52 of the metering member 25 are received between the pairs of splines 94 that have a separation of greater than 90° (the width of the extensions 52 is too great for them to be received between the other pairs of splines 94). The tapered tips of the extensions 52, however, are received between the less widely spaced parts of the splines 94 at the other end of the bushing 92. In a similar manner, a second metering member 25a can be engaged with the wheel 90 from the other side, with the extensions 52 of that metering member 25a being received in the spaces between splines 94 that are not occupied by the extensions of the first metering member 25.

As illustrated in FIG. 2, and shown in part but in greater detail in FIG. 6, the assembly of metering members 25, 25a and wheel 90 is accommodated between a pair of complementary support members 100,100a, each of which incorporates the respective reservoir 22, 22a and the respective frustoconical recesses 26, 26a. The metering members 25, 25a are closely received within the frustoconical recesses 26, 26a, the metering members 25, 25a being urged into close abutment with those recesses by the compression spring 28.

The ends of the compression spring 28 are received within the circular sockets 51, 51a of the two metering members 25, 25a. Those two sockets 51,51a, together with the extensions 52, 52a, form a substantially complete enclosure for the spring 28, permitting little or no lateral movement of the spring 28. The metering members 25, 25a are effectively coupled to the wheel 90, so that rotation of the wheel 90 causes rotation of both metering members 25, 25a in unison. Nonetheless, because the metering members 25, 25a are not fixed to the wheel 90, movement of the metering members 25, 25a along their axis of rotation is restrained only by the frustoconical recesses 26, 26a in the support members 100,100a within which the metering members 25, 25a are received. The metering members 25, 25a are therefore pressed into close engagement with those recesses 26, 26a by the action of the compression spring 28.

As can be seen in FIG. 6, the wheel 90 fits closely within the mouth of the recess 26 in the support member 100. The periphery of the mouth of the recess is formed with eight ratchet formations 102, spaced equiangularly. The pawls 95 engage with a diametrically-opposed pair of such ratchet formations 102. The effect of this is that the wheel 90 is able to rotate in one sense only, namely clockwise (as viewed in FIG. 6). Thus, when the wheel 90 is rotated clockwise, the pawls 95 are displaced from the positions they occupy in FIG. 6 and are deformed inwardly by the wall of the recess within which the wheel 90 rotates. When the pawls 95 come into registration with the next pair of ratchet formations 102, they engage with those ratchet formations 102. When the pawls 95 are so engaged, rotation of the wheel 90 in the opposite sense (i.e. anti-clockwise in FIG. 6) is prevented.

The manner in which the wheel 90 is caused to rotate will now be explained with reference to FIGS. 7 and 8. An actuator 70 is, in the assembled inhaler 1, captivated between the two support members 100, 100a. The actuator 70 has an upstand 72 that is received in a channel 104 defined by corresponding juxtaposed surfaces of the support members 100, 100a. The actuator 70 is acted upon directly by the push-button 4, such that the actuator 70 is capable of reciprocating motion, being displaced downwardly when the push-button 4 is depressed, and returning to its rest position when the push-button 4 is released. A compression spring (not shown) acts between the lower end of the channel 104 and the actuator 70 to return the latter (and the push button 4) to its rest position.

The lower end of the actuator 70 is formed with a limb 74 that acts on one of the lugs 93 on the wheel 90. In FIG. 8, it is the lug 93 at approximately the “three o'clock” position on the wheel 90 that is engaged by the limb 74 (shown in broken lines in FIG. 8) on the downstroke of the actuator 70.

The limb 74 presses the lug 93 downwards, causing the wheel 90 to rotate through 45° and displacing the pawls 95 from the positions shown in FIG. 6 to the circumferentially next ratchet formation 102. The length of the downstroke of the actuator 70 is sufficient to index the wheel 90 through 45° in that manner, the actuator 70 then returning along its upstroke to its rest position. On the upstroke, the limb 74 rides over the ramped part of the next following lug 93 and comes to rest adjacent to the flat top of that lug 93, so that repeated depression of the push button 4, and hence of the actuator 70, would index the wheel 90 through another 45°.

As noted above, the effect of each actuation is to rotate the wheel 90, and hence both metering members 25, 25a, through 45°. This displaces corresponding metering depressions 27, 27a in the respective metering members 25, 25a from a filling position, at which the metering depressions 27, 27a are gravity fed with medicament from the respective reservoirs 22, 22a, to a dispensing position from which the doses of medicament can be inhaled. At the same time, the next metering depressions 27, 27a are brought into registration with the reservoirs 22, 22a. Because reverse rotation of the wheel 90 (and hence of the metering members 25, 25a) is prevented by engagement of the pawls 95 with the ratchet formations 102, the metering members 25, 25a are held at the filling position. Any tendency for the metering depressions 27, 27a to be slightly displaced from that position, for instance due to frictional or other forces during the upstroke of the actuator 70 as it returns to its rest position, is thereby eliminated. This improves the uniformity of the doses of the two medicaments 21, 21 a dispensed from the inhaler 1 during repeated use.

1. A medicament delivery device comprising a metering member mounted for rotation adjacent a reservoir containing a bulk quantity of medicament, the metering member being adapted when at a filling position to volumetrically meter a desired dose of medicament from the reservoir and by rotation of the metering member in a first sense to transfer said dose to a dispensing position at which the dose can be dispensed from the device, the metering member and/or a component to which the metering member is operably linked being provided with formations effective to prevent rotation of the metering member from the filling position in the sense opposite to the first sense, the formations effective to prevent rotation of the metering member from the filling position in the sense opposite to the first sense constituting a ratchet-type mechanism, the metering member and/or the component to which the metering member is operably linked on the one hand and an adjacent surface of the device on the other hand being provided with ratchet and pawl formations, wherein the metering member and/or the component to which the metering member is operably linked is provided with one or more pawls that engage, when the metering member is in the filling position, with corresponding ratchet formations to prevent reverse rotation of the metering member.

2. A medicament delivery device according to claim 1, which is an inhaler.

3. A medicament delivery device according to claim 2, which is a dry powder inhaler.

4. A medicament delivery device according to any preceding claim, which is to dispense unit doses of a single medicament.

5. A medicament delivery device according to any one of claims 1 to 3, which is to dispense unit doses of a formulation containing two or more medicaments.

6. A medicament delivery device according to any one of claims 1 to 3, which is to dispense unit doses of two or more medicaments from separate reservoirs within the device, those unit doses then being administered to the patient simultaneously.

7. A medicament delivery device according to any preceding claim, wherein operation of the device is brought about by means of a push-button or the like, which acts upon an actuator that undergoes a reciprocating motion.

8. A medicament delivery device according to claim 7, wherein the actuator acts directly on the metering member.

9. A medicament delivery device according to claim 7, wherein the actuator acts upon a component that is operably linked to the metering member.

10. A medicament delivery device according to claim 9, wherein the device contains more than one reservoir of medicament containing different medicaments that are to be administered together.

11. A medicament delivery device according to claim 10, wherein each reservoir is associated with a separate metering member, and the metering members are coupled to a drive component such that rotation of the drive component causes rotation of the metering members.

12. A medicament delivery device according to claim 11, wherein the device contains two reservoirs of medicament, with first and second metering members, and the drive component has the form of a wheel mounted between the first and second metering members for rotation about a common axis.

13. A medicament delivery device according to claim 12, wherein rotation of the wheel is brought about by engagement of the actuator with a plurality of lugs that are equiangularly spaced around the wheel.

14. A medicament delivery device according to claim 12 or claim 13, wherein the wheel is provided with one or more pawls at its periphery.

15. A medicament delivery device according to claim 14, wherein the wheel is provided with two pawls.

16. A medicament delivery device according to claim 14 or claim 15, wherein the pawls are resilient and extend slightly from the circumference of the wheel.

17. A medicament delivery device according to any one of claims 14 to 16, wherein a number of ratchet formations are formed in a surface of the device adjacent to the periphery of the wheel.

18. A medicament delivery device according to claim 17, wherein the wheel rotates within a generally circular recess formed in a support component that holds the metering member, and the ratchet formations are formed in the wall of the circular recess, which is spaced from the edge of the wheel sufficiently to permit rotation in the first sense but is sufficiently close to the wheel to permit engagement of the pawl(s) with the ratchet formations whenever a metering cup is moved into the filling position.

19. A medicament delivery device as claimed in any preceding claim, which is charged with one or more medicaments selected from β2-agonists, eg fenoterol, formoterol, pirbuterol, reproterol, rimiterol, salbutamol, salmeterol and terbutaline; non-selective beta-stimulants such as isoprenaline; xanthine bronchodilators, eg theophylline, aminophylline and choline theophyllinate; anticholinergics, eg ipratropium bromide, oxitropium and tiotropium; mast cell stabilisers, eg sodium cromoglycate and ketotifen; bronchial anti-inflammatory agents, eg nedocromil sodium; and steroids, eg beclomethasone, fluticasone, budesonide, flunisolide, triamcinolone, mometasone and ciclesonide; and/or salts or derivatives thereof.

20. A medicament delivery device as claimed in any preceding claim, which is charged with a combination of medicaments selected from beclomethasone dipropionate and formoterol; beclomethasone dipropionate and salmeterol; fluticasone and formoterol; fluticasone and salmeterol; budesonide and formoterol; budesonide and salmeterol; flunisolide and formoterol; flunisolide and salmeterol; ciclesonide and salmeterol; ciclesonide and formoterol; mometasone and salmeterol; and mometasone and formoterol.
